(19) United States
(12) Patent Application Publication
(10) Pub. No.: US 2008/0139490 A1
(43) Pub. Date: Jun. 12, 2008

(54) Crystalline forms of valrubicin and processes for their preparation

(76) Inventors: Francesca Scarpitta, Ivrea (TO) (IT); Csilla Nemethne Racz, Tiszavasvari (HU)

Correspondence Address: KENYON & KENYON LLP, ONE BROADWAY, NEW YORK, NY, 10004, US

(21) Appl. No.: 11/904,066

(22) Filed: Sep. 25, 2007

Related U.S. Application Data
(60) Provisional application No. 60/847,588, filed on Sep. 26, 2006.
(60) Provisional application No. 60/853,504, filed on Oct. 19, 2006.

(51) Int. Cl. A61K 31/704; C07H 15/252; A61P 35/00

(52) U.S. Cl. 514/34; 536/6.4

(57) ABSTRACT

Provided are polymorphic forms of valrubicin and processes for their preparation.

### CROSS-REFERENCE TO RELATED APPLICATIONS

This application claims the benefit of U.S. provisional application Ser. Nos. 60/847,588, filed Sep. 26, 2006 and 60/853,504, filed Oct. 19, 2006, hereby incorporated by reference.

### FIELD OF THE INVENTION

The invention encompasses polymorphic forms of Valrubicin, processes for their preparation, and pharmaceutical compositions thereof.

### BACKGROUND OF THE INVENTION

Valrubicin, ((2S-cis)-2-[1,2,3,4,6,11-hexahydro-2,5,12-trihydroxy-7 methoxy-6,11-dioxo-[[4 2,3,6-trideoxy-3-[(trifluoroacetyl)-amino-α-L-lyxo-hexopyranosyl]oxyl]-2-naphthacenyl]-2-oxoethyl pentanoate, has the molecular formula C₃₄H₃₆F₃NO₁₃, a molecular weight of 723.66, and the following chemical structure:

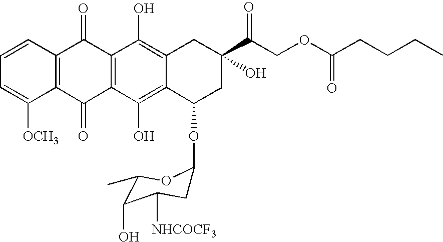

Valrubicin is reported to have been isolated as a orange or orange-red powder having a melting point range of 135-136° C. See Merck Index, pp. 1766-67, cpd. 9981 (13th ed. 2001). Valrubicin is a cytotoxic agent that is a semisynthetic analogue of the anthracycline doxorubicin, and is used to treat bladder cancer. Valrubicin is commercially available as VALSTAR® sterile solution for intravesical instillation, which is administered by direct infusion into the bladder.

The preparation of valrubicin was first reported in U.S. Pat. No. 4,035,566 (“'566 patent”). In the process of the '566 patent, valrubicin is prepared by reaction of 14-iodo-N-trifluoroacetyldaunomycin and sodium valerate in acetone. The crude product is isolated from the reaction mixture by extraction and precipitated from a mixture of chloroform and petroleum ether to yield valrubicin having a melting point of 135-136° C. See '566 patent, col. 3, 1.55 to col. 4, 1.15.

Another process for the preparation of valrubicin is disclosed in Organic Process Research and Development, 2005, 9, 818-821, wherein valrubicin is precipitated from a mixture of 2-butanone and petroleum ether as a red solid.

In addition, Synthetic Communications, 1999, 20, 3581, discloses the crystallization of valrubicin from a mixture of chloroform and hexane, providing a product having a melting point of 137-138° C.

The invention relates to the solid state physical properties of valrubicin. These properties can be influenced by controlling the conditions under which valrubicin is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (“TGA”) and differential scanning calorimetry (“DSC”) and can be used to distinguish some polymorphic forms from others. A particular polymorphic form may also give rise to distinct spectroscopic properties that may be detectable by powder X-ray crystallography, solid state ¹³C NMR spectrometry and infrared spectrometry.

One of the most important physical properties of a pharmaceutical compound, which can form polymorphs or solvates, is its solubility in aqueous solution, particularly the solubility in gastric juices of a patient. Other important properties relate to the ease of processing the form into pharmaceutical dosages, as the tendency of a powdered or granulated form to flow and the surface properties that determine whether crystals of the form will adhere to each other when compacted into a tablet.

The discovery of new polymorphic forms and solvates of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic. Therefore, there is a need for additional crystalline forms of valrubicin.

### SUMMARY OF THE INVENTION

In one embodiment, the invention encompasses a crystalline form of valrubicin characterized by data selected from the group consisting of at least one of: a powder X-ray diffraction pattern having peaks at about 6.4, 9.9 and 13.2° 2θ±0.2° 2θ; a powder X-ray diffraction pattern as depicted in FIG. 5; a Fourier-transform infrared spectrum having peaks at about 3544, 1732, and 1009 cm⁻¹; and a Fourier-transform infrared spectrum as depicted in FIG. 6.

In another embodiment, the invention encompasses a process for preparing a crystalline form of valrubicin characterized by data selected from the group consisting of at least one of: a powder X-ray diffraction pattern having peaks at about 6.4, 9.9 and 13.2° 2θ0±0.2° 2θ; a powder X-ray diffraction pattern as depicted in FIG. 5; a Fourier-transform infrared spectrum having peaks at about 3544, 1732, and 1009 cm⁻¹; and a Fourier-transform infrared spectrum as depicted in FIG. 6, comprising providing a suspension of Valrubicin in a mixture of a solvent selected from the group consisting of: dichloromethane, acetone, acetonitrile, methyl ethyl ketone, methylisobutyl ketone and an anti-solvent selected from the group consisting of: diisopropylether, and methyl-tert-butyl ether; and maintaining the suspension at a temperature of about 45° C. to 60° C. to obtain the above crystalline Valrubicin.

In another embodiment, the invention encompasses a crystalline form of valrubicin characterized by data selected from the group consisting of at least one of: a powder X-ray diffraction pattern having peaks at 3.9, 4.8 and 25.9° 2θ±0.2° 2θ; a powder X-ray diffraction pattern as depicted in FIG. 1; a Fourier-transform infrared spectrum having peaks at about 1724, 1415, and 1019 cm⁻¹; and a Fourier-transform infrared spectrum as depicted in FIG. 2.

In another embodiment, the invention encompasses a process for preparing a crystalline form of valrubicin characterized by data selected from the group consisting of at least one of: a PXRD pattern having peaks at 3.9, 4.8 and 25.9° 2θ±0.2° 2θ; a PXRD pattern as depicted in FIG. 1; a FT-IR spectrum having peaks at about 1724, 1415, and 1019 cm⁻¹, and an FT-IR spectrum as depicted in FIG. 2 by providing a suspension of Valrubicin in a mixture of a solvent selected from the group consisting of: dichloromethane, acetone, acetonitrile, methyl ethyl ketone, methylisobutyl ketone and an anti-solvent selected from the group consisting of: diisopropylether, and methyl-tert-butyl ether, and maintaining the suspension at a temperature of about 0° C. to 40° C. to obtain the above-described crystalline form of Valrubicin.

In another embodiment, the invention encompasses a pharmaceutical composition comprising at least one of the above-described crystalline forms of valrubicin, and at least one pharmaceutically acceptable excipient.

In another embodiment, the invention encompasses a process for preparing a pharmaceutical composition comprising at least one of the above-described crystalline forms of valrubicin, and at least one pharmaceutically acceptable excipient.

In another embodiment, the invention encompasses a method of treating bladder cancer comprising administering a therapeutically effective amount of the pharmaceutical composition to a patient in need thereof.

### BRIEF DESCRIPTION OF THE DRAWINGS

FIG. 1 illustrates a powder X-ray diffraction (“PXRD”) pattern of a crystalline form of valrubicin characterized by data selected from the group consisting of at least one of: a PXRD pattern having peaks at 3.9, 4.8 and 25.9° 2θ±0.2° 2θ; a PXRD pattern as depicted in FIG. 1; a FT-IR spectrum having peaks at about 1724, 1415, and 1019 cm⁻¹; and an FT-IR spectrum as depicted in FIG. 2.

FIG. 2 illustrates a FT-IR spectrum of a crystalline form of valrubicin characterized by data selected from the group consisting of at least one of: a PXRD pattern having peaks at 3.9, 4.8 and 25.9° 2θ±0.2° 2θ; a PXRD pattern as depicted in FIG. 1; a FT-IR spectrum having peaks at about 1724, 1415, and 1019 cm⁻¹; and an FT-IR spectrum as depicted in FIG. 2.

FIG. 3 illustrates a differential scanning calorimetry (“DSC”) curve of a crystalline form of valrubicin characterized by data selected from the group consisting of at least one of: a PXRD pattern having peaks at 3.9, 4.8 and 25.9° 2θ±0.2° 2θ; a PXRD pattern as depicted in FIG. 1; a FT-IR spectrum having peaks at about 1724, 1415, and 1019 cm⁻¹; and an FT-IR spectrum as depicted in FIG. 2.

FIG. 4 illustrates a microscope view of a crystalline form of valrubicin characterized by data selected from the group consisting of at least one of: a PXRD pattern having peaks at 3.9, 4.8 and 25.9° 2θ±0.2° 2θ; a PXRD pattern as depicted in FIG. 1; a FT-IR spectrum having peaks at about 1724, 1415, and 1019 cm⁻¹; and an FT-IR spectrum as depicted in FIG. 2.

FIG. 5 illustrates a PXRD pattern of a crystalline form of valrubicin characterized by data selected from the group consisting of at least one of: a PXRD pattern having peaks at about 6.4, 9.9 and 13.2° 2θ±0.2° 2θ; a PXRD pattern as depicted in FIG. 5; an FT-IR spectrum having peaks at about 3544, 1732, and 1009 cm⁻¹; and an FT-IR spectrum as depicted in FIG. 6.

FIG. 6 illustrates a FT-IR spectrum of a crystalline form of valrubicin characterized by data selected from the group consisting of at least one of: a PXRD pattern having peaks at about 6.4, 9.9 and 13.2° 2θ±0.2° 2θ; a PXRD pattern as depicted in FIG. 5; an FT-IR spectrum having peaks at about 3544, 1732, and 1009 cm⁻¹; and an FT-IR spectrum as depicted in FIG. 6.

FIG. 7 illustrates a microscope view of a crystalline form of valrubicin characterized by data selected from the group consisting of at least one of: a PXRD pattern having peaks at about 6.4, 9.9 and 13.2° 2θ±0.2° 2θ; a PXRD pattern as depicted in FIG. 5; an FT-IR spectrum having peaks at about 3544, 1732, and 1009 cm⁻¹; and an FT-IR spectrum as depicted in FIG. 6.

FIG. 8 illustrates a DSC curve of crystalline form of valrubicin characterized by data selected from the group consisting of at least one of: a PXRD pattern having peaks at about 6.4, 9.9 and 13.2° 2θ±0.2° 2θ; a PXRD pattern as depicted in FIG. 5; an FT-IR spectrum having peaks at about 3544, 1732, and 1009 cm⁻¹; and an FT-IR spectrum as depicted in FIG. 6.

### DETAILED DESCRIPTION OF THE INVENTION

The invention encompasses crystalline forms of valrubicin, processes for preparation thereof and pharmaceutical compositions thereof.

The time periods described herein are time periods suitable for laboratory-scale preparations. One of ordinary skill in the art understands that suitable time periods will vary based upon the amounts of reagents present, and can adjust the time periods accordingly.

One of ordinary skill in the art is aware that there is a certain amount of experimental error inherent in powder X-ray diffraction (“PXRD”) techniques. See, e.g., U.S. PHARMACOPEIA, 387-89 (30th ed. 2007), hereby incorporated by reference. As to individual peaks, peak positions are reported over a range of ±0.2° 2θ to account for this experimental error. As to PXRD patterns in their entirety, the term “as depicted” in a particular figure is meant to account for this experimental error, as well as for variations in peak position and intensity due to factors such as, for example, variations in sample preparation, instrumentation, and the skill of the operator of the instrument. A PXRD pattern “as depicted” in a particular figure means that one of ordinary skill in the art, understanding the experimental error involved in powder X-ray diffraction techniques, would determine that the PXRD pattern corresponds to the same crystalline structure as the PXRD pattern depicted in the figure.

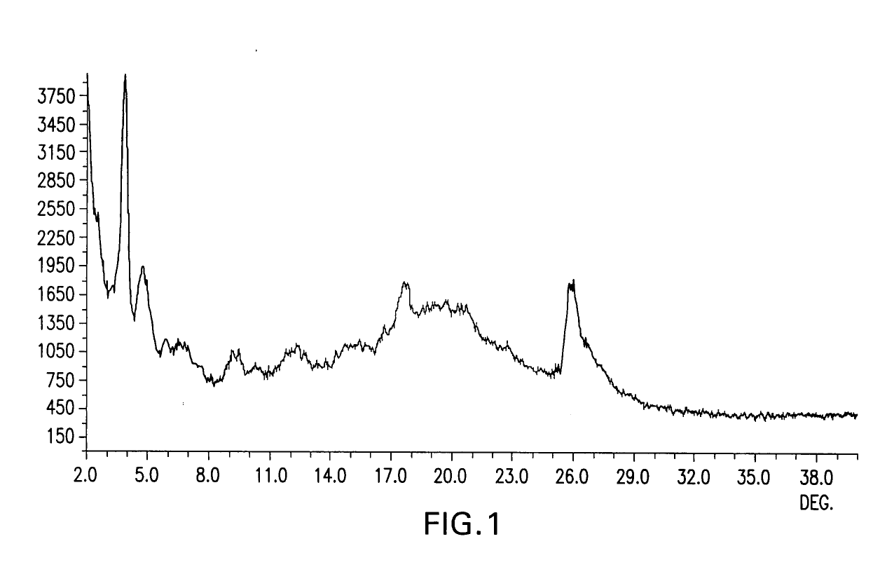

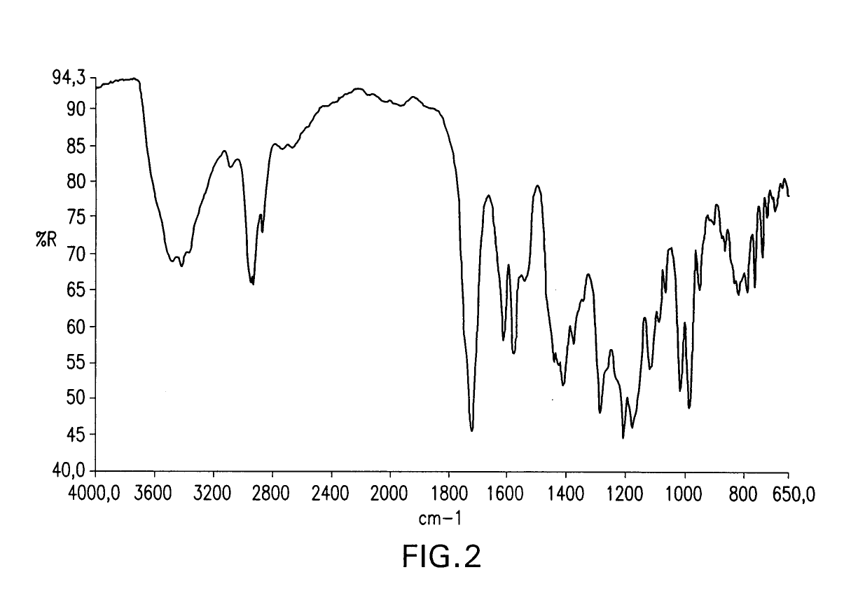

In one embodiment, the invention encompasses crystalline valrubicin characterized by data selected from the group consisting of at least one of: a PXRD pattern having peaks at 3.9, 4.8 and 25.9° 2θ±0.2° 2θ; a PXRD pattern as depicted in FIG. 1; a FT-IR spectrum having peaks at about 1724, 1415, and 1019 cm⁻¹, and an FT-IR spectrum as depicted in FIG. 2.

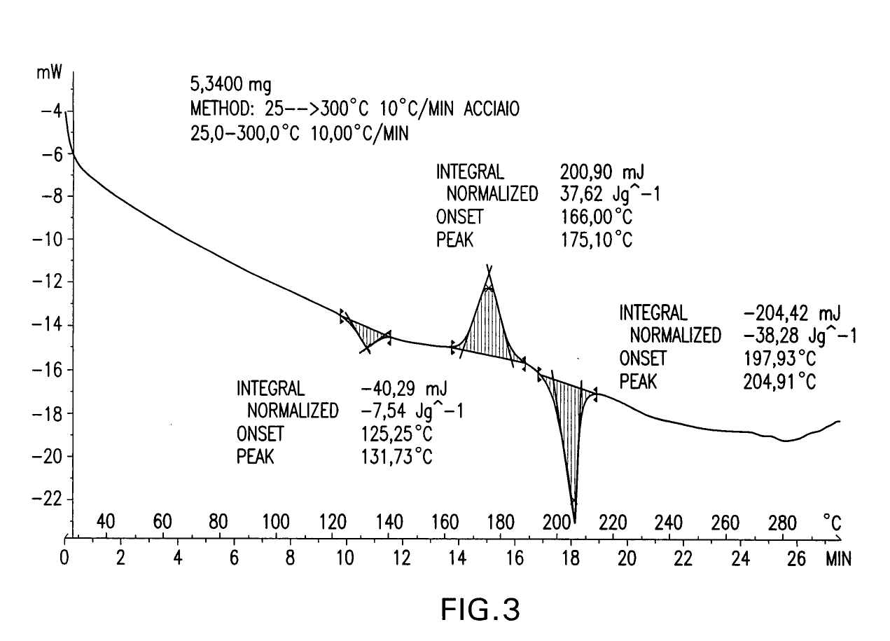

The crystalline form may be further characterized by data selected from the group consisting of at least one of: an FT-IR spectrum having peaks at about 1616, 1582, 1289, 1209, 1181, 987, 764 and 740 cm⁻¹; a DSC thermogram having an endothermic peak at about 130° C., an exothermic peak at about 175° C., and an endothermic peak at about 206° C.; a DSC thermogram as depicted in FIG. 3.

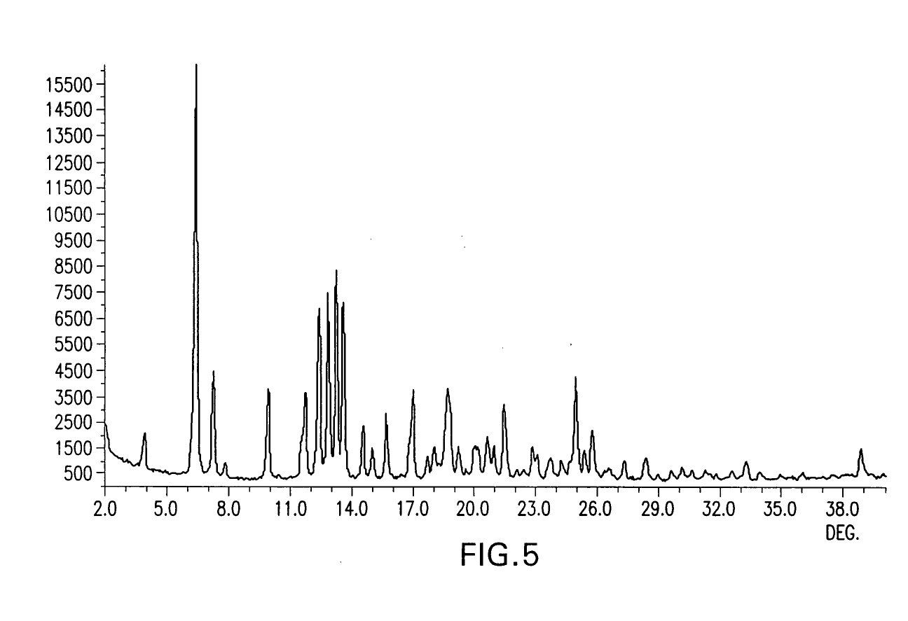

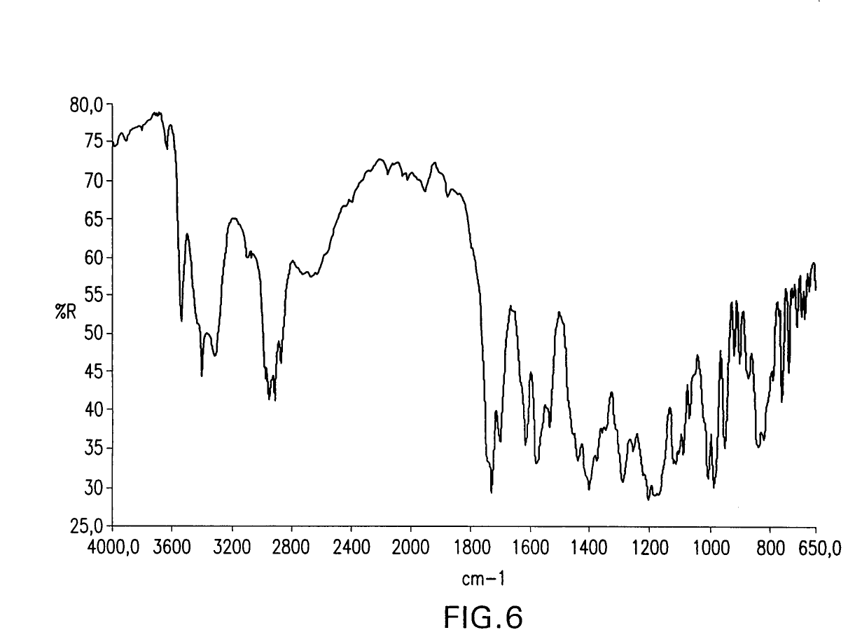

Preferably the crystalline form has no more than 50%, more preferably no more than about 25%, even more preferably not more than 20%, even more preferably not more than about 15%, and most preferably not more than about 10% of crystalline valrubicin characterized by data selected from the group consisting of at least one of: a PXRD pattern having peaks at about 6.4, 9.9 and 13.2° 2θ±0.2° 2θ; a PXRD pattern as depicted in FIG. 5; an FT-IR spectrum having peaks at about 3544, 1732, and 1009 cm⁻¹; and an FT-IR spectrum as depicted in FIG. 6. Typically, the content of crystalline valrubicin having a PXRD pattern having peaks at 6.4, 9.9 and 13.2° 2θ±0.2° 2θ in the above form is measured by percent by weight. Preferably, the content is determined by PXRD. The determination by PXRD can be done using the peak at 6.4 degrees two-theta ±0.2 degrees two-theta.

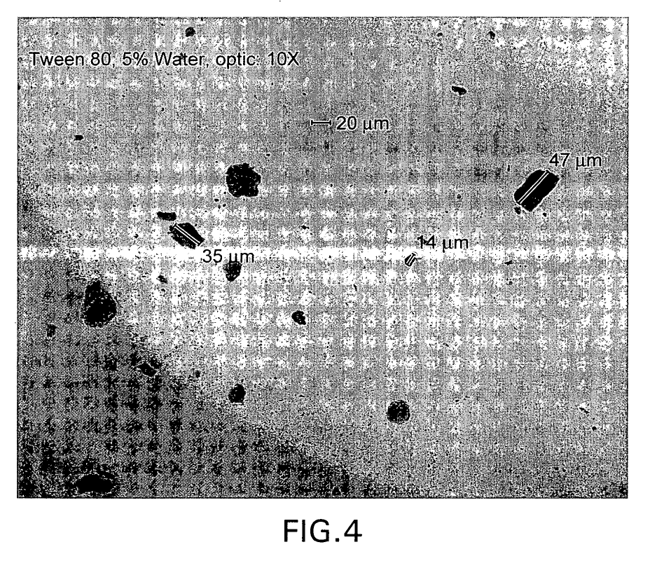

The above crystalline Valrubicin has irregular shaped particles, as illustrated in FIG. 4. The morphology of an active pharmaceutical ingredient (“API”) typically affects handling of the API during milling and drug product manufacturing. Irregular-shaped particles are advantageous because they generally have better flowability than, for example, needle-shaped particles.

In another embodiment, the invention encompasses a process for preparing the above-described crystalline form of valrubicin characterized by data selected from the group consisting of at least one of: a PXRD pattern having peaks at 3.9, 4.8 and 25.9° 2θ±0.2° 2θ; a PXRD pattern as depicted in FIG. 1; a FT-IR spectrum having peaks at about 1724, 1415, and 1019 cm⁻¹, and an FT-IR spectrum as depicted in FIG. 2 by providing a suspension of Valrubicin in a mixture of a solvent selected from the group consisting of: dichloromethane, acetone, acetonitrile, methyl ethyl ketone, methylisobutyl ketone and an anti-solvent selected from the group consisting of: diisopropylether, and methyl-tert-butyl ether, and maintaining the suspension at a temperature of about 0° C. to 40° C. to obtain the above-described crystalline form of Valrubicin.

Preferably, the solvent is acetone, acetonitrile, or dichloromethane, and more preferably dichloromethane.

Preferably, the anti-solvent is diisopropylether.

One particularly preferred combination of solvent and anti-solvent is dichloromethane and diisopropyl ether, respectively.

Preferably, the suspension of Valrubicin is provided by dissolving the Valrubicin in the solvent and admixing the solution with the anti-solvent. Preferably, the anti-solvent is added to the solution.

Preferably, the solution and the anti-solvent are admixed at a temperature of about 0° C. to about 40° C., and more preferably at a temperature of about 15° C. to about 25° C.

Preferably, the anti-solvent is present in the suspension in an amount of at least 5 volumes per volume of the solvent, more preferably in an amount of about 5 to about 10 volumes per volume of the solvent, and most preferably in an amount of about 6 to about 7 volumes per volume of the solvent.

The anti-solvent may be added to the solution portion-wise or drop-wise, and is preferably added drop-wise. When the anti-solvent is added portion-wise, it is added in at least one portion, and preferably in two or more portions. Preferably, the size of the portion is about 1 to about 6.5 volumes of the anti-solvent per volume of solvent and more preferably about 1.25 to about 1.5 volumes. When the anti-solvent is added drop-wise, it is preferably added over a period of about 1 to about 6 hours, and more preferably over a period of about 2 to about 3 hours.

Preferably, the suspension is maintained at a temperature of about 10° C. to about 30° C., and more preferably about 15° C. to about 25° C., to form the crystalline form of valrubicin. Preferably, the suspension is maintained for about 1 to about 12 hours and more preferably for about 3 to about 4 hours.

The crystalline form of valrubicin may be recovered from the suspension by any method known to one of ordinary skill in the art. Suitable methods include, but are not limited to, filtering the crystalline form of valrubicin from the suspension, optionally washing the crystalline form of valrubicin with the anti-solvent, and drying the crystalline form of valrubicin. Typically, the crystalline form of valrubicin is dried at a temperature of about 40° C. to about 65° C. Preferably, the crystalline form of valrubicin is dried with heating under vacuum, and more preferably at a pressure of about 18 mbar.

In yet another embodiment, the invention encompasses crystalline valrubicin characterized by data selected from the group consisting of at least one of: a PXRD pattern having peaks at about 6.4, 9.9 and 13.2° 2θ±0.2° 2θ; a PXRD pattern as depicted in FIG. 5; an FT-IR spectrum having peaks at about 3544, 1732, and 1009 cm⁻¹, and an FT-IR spectrum as depicted in FIG. 6.

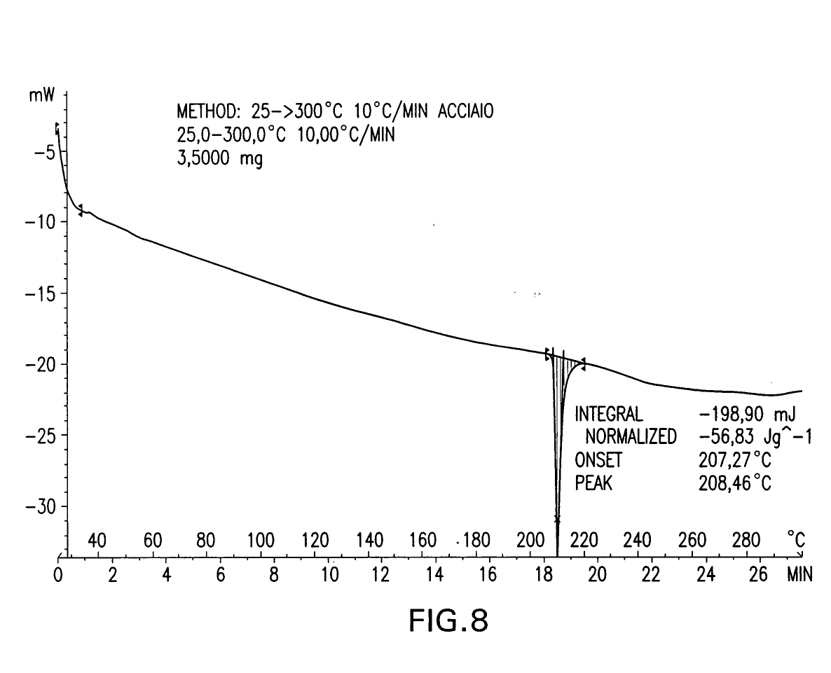

The crystalline valrubicin may be further characterized by data selected from the group consisting of at least one of: a PXRD pattern having peaks at 7.2, 12.4, 12.8, 13.6, 21.4 and 24.9° 2θ±0.2° 2θ; an FT-IR spectrum having peaks at about 3405, 1702, 1616, 1582, 1406, 1293, 990, 762 and 739 cm⁻¹; a DSC thermogram having an endothermic peak at about 208° C.; and a DSC thermogram as depicted in FIG. 8.

Preferably the crystalline form has no more than 50%, more preferably no more than 25%, even more preferably not more than 20%, even more preferably not more than 15%, and most preferably not more than 10% of crystalline valrubicin characterized by data selected from the group consisting of at least one of: a PXRD pattern having peaks at 3.9, 4.8 and 25.9° 2θ±0.2° 2θ; a PXRD pattern as depicted in FIG. 1; a FT-IR spectrum having peaks at about 1724, 1415, and 1019 cm⁻¹ and an FT-IR spectrum as depicted in FIG. 2. Typically, the content of crystalline valrubicin having a PXRD pattern having peaks at 3.9, 4.8 and 25.9° 2θ±0.2° 2θ in the above form is measured by percent by weight. Preferably, the content is determined by PXRD. The determination by PXRD can be done using the peak at 4.8 degrees two-theta +0.2 degrees two-theta.

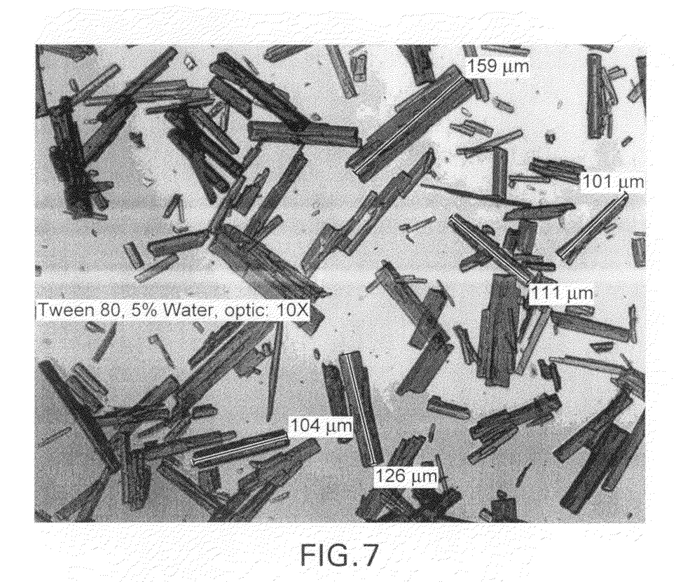

The above-described crystalline valrubicin characterized by data selected from the group consisting of at least one of: a PXRD pattern having peaks at about 6.4, 9.9 and 13.20 2θ±0.2° 2θ; a PXRD pattern as depicted in FIG. 5; an FT-IR spectrum having peaks at about 3544, 1732, and 1009 cm⁻¹, and an FT-IR spectrum as depicted in FIG. 6 is preferably desirable at least because it is thermodynamically stable, as evidenced by its high melting point. This crystalline form of valrubicin has a melting point of about 208° C., while the valrubicin obtained by the process disclosed in U.S. Pat. No. 4,035,566 is reported to have a melting point range of 135° C. to 136° C. and the valrubicin obtained by the process disclosed in Synthetic Communications, 1999, 20, 3581 is reported to have a melting point range of 137° C. to 138° C. In addition, this crystalline valrubicin has rod-shaped crystals, as illustrated in FIG. 7, which are easy to filter even when produced on an industrial scale.

In another embodiment, the invention encompasses a process for preparing the above-described crystalline valrubicin characterized by data selected from the group consisting of at least one of: a PXRD pattern having peaks at about 6.4, 9.9 and 13.2° 2θ±0.2° 2θ; a PXRD pattern as depicted in FIG. 5; an FT-IR spectrum having peaks at about 3544, 1732, and 1009 cm⁻¹, and an FT-IR spectrum as depicted in FIG. 6 by providing a suspension of Valrubicin in a mixture of a solvent selected from the group consisting of: dichloromethane, acetone, acetonitrile, methyl ethyl ketone, methylisobutyl ketone and an anti-solvent selected from the group consisting of: diisopropylether, and methyl-tert-butyl ether; and maintaining the suspension at a temperature of about 45° C. to 60° C. to obtain the above-described crystalline Valrubicin.

Preferred combinations of solvent and anti-solvent include acetone and methyl tert-butyl ether; acetonitrile and methyl tert-butyl ether; methyl ethyl ketone and methyl tert-butyl ether; dichloromethane and methyl tert-butyl ether; acetone and diisopropylether; acetonitrile and diisopropylether; methyl ethyl ketone and diisopropylether; methylisobutyl ketone and diisopropylether; and dichloromethane and diisopropylether. More preferred combinations of solvent and anti-solvent include acetone and diisopropylether; acetonitrile and diisopropylether; or dichloromethane and diisopropylether.

Preferably, the solvent is dichloromethane, acetone, or acetonitrile, and more preferably dichloromethane.

Preferably, the anti-solvent is diisopropylether.

Typically, the suspension is provided by dissolving the valrubicin in the solvent to form a solution and admixing the solution with the anti-solvent. Preferably, the valrubicin and solvent are maintained at a temperature of about 0° C. to about 30° C., and more preferably at a temperature of about 25° C. to about 30° C., to dissolve the valrubicin.

Preferably, the admixing is performed by adding the anti-solvent to the solution. When the anti-solvent is added to the solution, the addition is preferably done at a temperature of about 25° C. to about 40° C., and more preferably at a temperature of about 25° C. to about 30° C. When the solution is added to the anti-solvent, the addition is preferably done at a temperature of about 45° C. to 60° C. and preferably at a temperature of about 50° C. to about 60° C.

Optionally, the suspension can be provided by suspending Valrubicin in a mixture of the solvent and anti-solvent; wherein the solvent and anti-solvent are combined prior to suspending Valrubicin in their mixture. In such process, the preferred combinations of solvent and anti-solvent include acetone and methyl-tert-butyl ether, acetonitrile and methyl-tert-butyl ether, methyl ethyl ketone and methyl-tert-butyl ether, dichloromethane and methyl-tert-butyl ether, acetone and diisopropylether, acetonitrile and diisopropylether, methyl ethyl ketone and diisopropylether, methylisobutyl ketone and diisopropylether, or dichloromethane and diisopropylether. More preferably, the mixture of solvent and anti-solvent comprises: acetone and diisopropylether, acetonitrile and diisopropylether, or dichloromethane and diisopropylether.

Preferably, the Valrubicin is suspended in the mixture of solvent and anti-solvent at a temperature of about 0° C. to about 30° C., and more preferably at about 25° C. to about 30° C.

Preferably, the anti-solvent is present in the suspension in an amount of at least 5 volumes per volume of the solvent, more preferably in an amount of about 5 to about 10 volumes per volume of the solvent, and most preferably in an amount of about 6 to about 7 volumes per volume of the solvent.

When the anti-solvent is added to the solution, it may be added either portion-wise or drop-wise, and is preferably added drop-wise. When the anti-solvent is added portion-wise, it is added in at least one portion, and preferably in two or more portions. Preferably, the size of the portion is about 1 to about 6.5 volumes of the anti-solvent per volume of solvent and more preferably about 1.25 to about 1.5 volumes. When the anti-solvent is added drop-wise, it is preferably added over a period of about 1 to about 6 hours, and more preferably over a period of about 2 to about 3 hours.

Preferably, the addition of the anti-solvent to the solution causes the formation of a suspension containing the crystalline form of valrubicin.

Preferably, the suspension is maintained at a temperature of about 50° C. to about 65° C., and more preferably about 50° C. to about 60° C., to form the crystalline form of valrubicin. Preferably, the suspension is maintained for about 0.5 to about 3 hours and more preferably for about 0.5 to about 1 hour.

The crystalline form of valrubicin may be recovered from the suspension by any method known to one of ordinary skill in the art. Suitable methods include, but are not limited to, filtering the crystalline form of valrubicin from the suspension, optionally washing the crystalline form of valrubicin with the anti-solvent, and drying the crystalline form of valrubicin. Typically, the crystalline form of valrubicin is dried at a temperature of about 40° C. to about 65° C. Preferably, the crystalline form of valrubicin is dried with heating under vacuum, and more preferably at a pressure of about 18 mbar.

In yet another embodiment, the invention encompasses a pharmaceutical composition comprising at least one of the above-described crystalline forms of valrubicin, and at least one pharmaceutically acceptable excipient.

Preferably, the crystalline form of valrubicin is prepared by one of the above-described processes.

The pharmaceutical composition may optionally contain other forms of valrubicin and/or additional active ingredients. The amount of valrubicin or other active ingredient present in the pharmaceutical composition should be sufficient to treat, ameliorate, or reduce the target condition.

The pharmaceutically acceptable excipient may be any excipient commonly known to one of skill in the art to be suitable for use in pharmaceutical compositions. Suitable pharmaceutically acceptable excipients include, but are not limited to, diluents, carriers, fillers, bulking agents, binders, disintegrants, disintegration inhibitors, absorption accelerators, wetting agents, lubricants, glidants, surface active agents, flavoring agents, and the like. Selection of excipients and the amounts to use can be readily determined by an experienced formulation scientist in view of standard procedures and reference works known in the art.

The pharmaceutical composition may be formulated into a solid dosage form. Suitable solid dosage forms include, but are not limited to, tablets, pills, powders, granules, capsules, suppositories, and the like. The choice of dosage form may depend, for example, on the age, sex, and symptoms of the patient.

In another embodiment, the invention encompasses a process for preparing a pharmaceutical composition comprising combining at least one of the above-described crystalline forms of valrubicin, with at least one pharmaceutically acceptable excipient. Preferably, the crystalline form of valrubicin is prepared by one of the above-described processes.

In another embodiment, the invention encompasses the use of at least one of the above-described crystalline forms of valrubicin in the manufacture of a pharmaceutical composition.

In another embodiment, the invention encompasses a method of treatment of bladder cancer comprising administering a therapeutically effective amount of a pharmaceutical composition comprising at least one of the above-described crystalline forms of valrubicin, and at least one pharmaceutically acceptable excipient to a patient in need thereof.

Having described the invention with reference to certain preferred embodiments, other embodiments will become apparent to one skilled in the art from consideration of the specification. The invention is further defined by reference to the following examples describing in detail the process and compositions of the invention. It will be apparent to those skilled in the art that many modifications, both to materials and methods, may be practiced without departing from the scope of the invention.

### EXAMPLES

### X-Ray Powder Diffraction (“PXRD”)

ARL X-ray powder diffractometer model X'TRA-030, Peltier detector, round standard aluminium sample holder with round zero background quartz plate was used. Scanning parameters: Range: 2-40 deg. 2 θ, continuous Scan, Rate: 3 deg./min. Copper radiation at a wavelength of 1.5418 Å was used. The accuracy of peak positions is defined as +/−0.2 degrees due to experimental differences like instrumentations, sample preparations etc.

### FT-IR Spectroscopy

Perkin-Elmer Spectrum One Spectrometer, at 4 cm-1 resolution with 20 scans, in the range of 4000-650 cm⁻¹. Samples were analysed in KBr with Drift technique. The spectra were recorded using KBr as a background.

### Differential Scanning Calorimetry (“DSC”)

DSC 822, Mettler Toledo, Sample weight: 3-5 mg. Heating rate: 10° C./min., Number of holes of the crucible: 2. Scan range: 25-300° C., 10° C./minutes heating rate.

### Example 1

Preparation of crystalline valrubicin characterized by data selected from the group consisting of at least one of: a PXRD pattern having peaks at 3.9, 4.8 and 25.9° 2θ±0.2° 2θ; a PXRD pattern as depicted in FIG. 1; a FT-IR spectrum having peaks at about 1724, 1415, and 1019 cm⁻¹, and an FT-IR spectrum as depicted in FIG. 2.

0.8 g of valrubicin were dissolved at room temperature in 4 ml of dichloromethane (“DCM”) to form a solution. The solution was then cooled to 15° C. and 5 ml of diisopropyl ether (“DIPE”) were added drop-wise to the solution with stirring. After about 20 minutes precipitation was observed. The suspension was then maintained with stirring for 1 hour. Then, about 15 ml of DIPE was added portion-wise to the suspension over a period of two hours (three 5 ml portions of DIPE were slowly added to the suspension every 15 minutes) at 15° C. and the suspension was stirred. The suspension was then left stirring for an additional 30 minutes. Finally, an additional 6 ml of DIPE were added drop-wise (for a total amount of 32.5 volumes of DIPE per gram of valrubicin). The suspension was then maintained at 15° C. with stirring for an additional 4 hours. The solid was then filtered from the suspension with a Buchner funnel. The red solid thus obtained was washed with 5 ml of DIPE and dried under vacuum at 40° C. overnight, to afford 0.4 g of crystalline valrubicin.

### Example 2

Preparation of crystalline valrubicin characterized by data selected from the group consisting of at least one of: a PXRD pattern having peaks at 3.9, 4.8 and 25.9° 2θ±0.2° 2θ; a PXRD pattern as depicted in FIG. 1; a FT-IR spectrum having peaks at about 1724, 1415, and 1019 cm⁻¹, and an FT-IR spectrum as depicted in FIG. 2.

0.8 g of valrubicin were dissolved at room temperature in 4 ml of dichloromethane and 5 ml of diisopropylether are added drop-wise to the solution with stirring. After about 15 minutes, precipitation was observed. The obtained suspension was then maintained with stirring for 1 hour. Then, about 15 ml of DIPE was added portion-wise to the suspension over a period of two hours (three 5 ml portions of DIPE were slowly added to the suspension every 15 minutes) at 15° C. and the suspension was stirred. The suspension was then left stirring for an additional 30 minutes. Finally, an additional 6 ml of DIPE were added dropwise (for a total amount of 32.5 volumes of DIPE per gram of valrubicin). The suspension was then maintained at 15° C. with stirring for an additional 4 hours. The solid was then filtered from the suspension with a Buchner funnel. The red solid thus obtained was washed with 5 ml of DIPE and dried under vacuum at 40° C. overnight, to afford 0.6 g of crystalline valrubicin.

### Example 3

Preparation of crystalline valrubicin characterized by data selected from the group consisting of at least one of: a PXRD pattern having peaks at about 6.4, 9.9 and 13.2° 2θ±0.2° 2θ; a PXRD pattern as depicted in FIG. 5; an FT-IR spectrum having peaks at about 3544, 1732, and 1009 cm⁻¹, and an FT-IR spectrum as depicted in FIG. 6 (DCM/DIPE-Direct Addition)

10.0 g of valrubicin were loaded in a 1 L reactor and dissolved in 50 ml of DCM at 25° C. 325 ml of DIPE were then added drop-wise to the solution over a period of about 3 hours. After about 70-80 ml of DIPE were added, precipitation occurred. Once the addition was complete, the obtained suspension was heated to 60° C. over about 2.5 hours. When the suspension reached a temperature of 45° C., a change in the color was noticed and the suspension become darker. The suspension was then maintained at 60° C. with stirring for half an hour. Then, the suspension was cooled to 25° C. over 3 hours. The red solid thus obtained was filtered from the suspension in gooch P3 and washed with 20 ml of DIPE. The solid was then dried under vacuum at 40° C. for 7 hours, to afford 9.37 g of crystalline valrubicin.

### Example 4

Preparation of crystalline valrubicin characterized by data selected from the group consisting of at least one of: a PXRD pattern having peaks at about 6.4, 9.9 and 13.2° 2θ±0.2° 2θ; a PXRD pattern as depicted in FIG. 5; an FT-IR spectrum having peaks at about 3544, 1732, and 1009 cm⁻¹, and an FT-IR spectrum as depicted in FIG. 6 (DCM/DIPE—Inverse Addition)

1.5 g of valrubicin were dissolved in 7.5 ml of dichloromethane at 25° C. The solution was then added drop-wise, over about one hour, to 48.7 ml of diisopropyl ether and heated to 60° C. After the addition was complete, the obtained suspension was stirred at 60° C. for about one hour. Then, the suspension was cooled to 25° C. over 3 hours. The red solid thus obtained was filtered from the suspension in gooch P3 and washed with 10 ml of DIPE. The solid was then dried under vacuum at 40° C. for 7 hours, to afford 1.38 g of crystalline valrubicin.

### Example 5

Preparation of crystalline valrubicin characterized by data selected from the group consisting of at least one of: a PXRD pattern having peaks at about 6.4, 9.9 and 13.2° 2θ±0.2° 2θ: a PXRD pattern as depicted in FIG. 5; an FT-IR spectrum having peaks at about 3544, 1732, and 1009 cm⁻¹, and an FT-IR spectrum as depicted in FIG. 6 (acetone/DIPE)

1.5 g of valrubicin were dissolved at 25° C. in 7.5 ml of acetone to form a solution. 48.7 ml of diisopropyl ether were then added drop-wise to the solution at 25° C. over 1 hour. Precipitation was observed. After the addition was complete, the obtained suspension was heated to 60° C. and maintained at 60° C. with stirring for half an hour. Then, the suspension was cooled to 25° C. over 3 hours. The red solid thus obtained was filtered from the suspension in gooch P3 and washed with 10 ml of DIPE. The solid was then dried under vacuum at 40° C. for 7 hours, to afford 1.13 g of crystalline valrubicin.

### Example 6

Preparation of crystalline valrubicin characterized by data selected from the group consisting of at least one of: a PXRD pattern having peaks at about 6.4, 9.9 and 13.2° 2θ±0.2° 2θ; a PXRD pattern as depicted in FIG. 5; an FT-IR spectrum having peaks at about 3544, 1732, and 1009 cm⁻¹, and an FT-IR spectrum as depicted in FIG. 6.

1.5 g of valrubicin were dissolved at 25° C. in 3.75 ml of acetonitrile to form a solution. 48.7 ml of diisopropyl ether were then added drop-wise to the solution at 25° C. over 1 hour. Precipitation was observed. After the addition was complete, the obtained suspension was heated to 60° C. over a period of 1 hour and maintained at 60° C. with stirring for half an hour. Then, the suspension was cooled to 25° C. over 3 hours. The red solid thus obtained was filtered from the suspension in gooch P3 and washed with 10 ml of DIPE. The solid was then dried under vacuum at 40° C. for 7 hours, to afford 1.17 g of crystalline valrubicin.

### Example 7

Preparation of crystalline valrubicin characterized by data selected from the group consisting of at least one of: a PXRD pattern having peaks at about 6.4, 9.9 and 13.2° 2θ±0.2° 2θ; a PXRD pattern as depicted in FIG. 5; an FT-IR spectrum having peaks at about 3544, 1732, and 1009 cm⁻¹, and an FT-IR spectrum as depicted in FIG. 6.

1.5 g of valrubicin were suspended in 7.5 ml of dichloromethane and 48.7 ml of diisopropyl ether at 25° C. The obtained suspension was then heated to 60° C. over a period of 1 hour and maintained at 60° C. with stirring for half an hour. Then, the suspension was cooled to 25° C. over 3 hours. The red solid thus obtained was filtered from the suspension in gooch P3 and washed with 10 ml of DIPE. The solid was then dried under vacuum at 40° C. for 7 hours, to afford 1.40 g of crystalline valrubicin.

What is claimed is:

1. A crystalline form of valrubicin characterized by data selected from the group consisting of at least one of: a powder X-ray diffraction pattern having peaks at about 6.4, 9.9 and 13.2° 2θ±0.2° 2θ; a powder X-ray diffraction pattern as depicted in FIG. 5; a Fourier-transform infrared spectrum having peaks at about 3544, 1732, and 1009 cm⁻¹; and a Fourier-transform infrared spectrum as depicted in FIG. 6.

2. The crystalline form of valrubicin of claim 1, characterized by a powder X-ray diffraction pattern having peaks at about 6.4, 9.9 and 13.2° 2θ±0.2° 2θ.

3. The crystalline form of valrubicin of claim 1, characterized by a Fourier-transform infrared spectrum having peaks at about 3544, 1732, and 1009 cm⁻¹.

4. The crystalline form of valrubicin of claim 1, characterized by a powder X-ray diffraction pattern as depicted in FIG. 5.

5. The crystalline form of valrubicin of claim 1, characterized by a Fourier-transform infrared spectrum as depicted in FIG. 6.

6. The crystalline form of valrubicin of claim 2, further characterized by a powder X-ray diffraction pattern having peaks at 7.2, 12.4, 12.8, 13.6, 21.4 and 24.9° 2θ±0.2° 2θ.

7. The crystalline form of valrubicin of claim 2, further characterized by a Fourier-transform infrared spectrum having peaks at about 3405, 1702, 1616, 1582, 1406, 1293, 990, 762 and 739 cm⁻¹.

8. The crystalline form of valrubicin of claim 1, further characterized by a differential scanning calorimetry thermogram having an endothermic peak at about 208° C.

9. The crystalline form of valrubicin of claim 8, further characterized by a differential scanning calorimetry thermogram as depicted in FIG. 8.

10. The crystalline form of valrubicin of claim 1, having no more than 50% of a crystalline form of valrubicin characterized by data selected from the group consisting of at least one of: a powder X-ray diffraction pattern having peaks at 3.9, 4.8 and 25.9° 2θ±0.2° 2θ; a powder X-ray diffraction pattern as depicted in FIG. 1; a Fourier-transform infrared spectrum having peaks at about 1724, 1415, and 1019 cm⁻¹; and a Fourier-transform infrared spectrum as depicted in FIG. 2.

11. A process for preparing a crystalline form of valrubicin characterized by data selected from the group consisting of at least one of: a powder X-ray diffraction pattern having peaks at about 6.4, 9.9 and 13.2° 2θ±0.2° 2θ; a powder X-ray diffraction pattern as depicted in FIG. 5; a Fourier-transform infrared spectrum having peaks at about 3544, 1732, and 1009 cm⁻¹; and a Fourier-transform infrared spectrum as depicted in FIG. 6, comprising providing a suspension of valrubicin in a mixture of a solvent selected from the group consisting of dichloromethane, acetone, acetonitrile, methyl ethyl ketone, methylisobutyl ketone and an anti-solvent selected from the group consisting of diisopropylether, and methyl-tert-butyl ether; and maintaining the suspension at a temperature of about 45° C. to 60° C. to obtain the crystalline form of valrubicin.

12. The process of claim 11, wherein the suspension is provided by dissolving the valrubicin in the solvent to form a solution, and admixing the solution with the anti-solvent to form the suspension.

13. The process of claim 11, wherein the suspension is provided by suspending the valrubicin in a mixture of the solvent and the anti-solvent, wherein the solvent and the anti-solvent are combined prior to suspending the valrubicin in their mixture.

14. The process of claim 11, wherein the solvent is dichloromethane, acetone, acetonitrile, or methyl ethyl ketone.

15. The process of claim 11, wherein the anti-solvent is diisopropyl ether.

16. The process of claim 11, wherein the suspension is maintained at a temperature of about 50° C. to about 60° C.

17. A pharmaceutical composition comprising a crystalline form of valrubicin characterized by data selected from the group consisting of at least one of: a powder X-ray diffraction pattern having peaks at about 6.4, 9.9 and 13.2° 2θ±0.2° 2θ; a powder X-ray diffraction pattern as depicted in FIG. 5; a Fourier-transform infrared spectrum having peaks at about 3544, 1732, and 1009 cm⁻¹; and a Fourier-transform infrared spectrum as depicted in FIG. 6; and at least one pharmaceutically acceptable excipient.

18. A method of treating bladder cancer comprising administering a therapeutically effective amount of a pharmaceutical composition comprising a crystalline form of valrubicin characterized by data selected from the group consisting of at least one of: a powder X-ray diffraction pattern having peaks at about 6.4, 9.9 and 13.2° 2θ±0.2° 2θ; a powder X-ray diffraction pattern as depicted in FIG. 5; a Fourier-transform infrared spectrum having peaks at about 3544, 1732, and 1009 cm⁻¹; and a Fourier-transform infrared spectrum as depicted in FIG. 6; and at least one pharmaceutically acceptable excipient to a patient in need thereof.

19. A crystalline form of valrubicin characterized by data selected from the group consisting of at least one of: a powder X-ray diffraction pattern having peaks at 3.9, 4.8 and 25.9° 2θ±0.2° 2θ; a powder X-ray diffraction pattern as depicted in FIG. 1; a Fourier-transform infrared spectrum having peaks at about 1724, 1415, and 1019 cm⁻¹; and a Fourier-transform infrared spectrum as depicted in FIG. 2.

20. The crystalline form of valrubicin of claim 19, characterized by a powder X-ray diffraction pattern having peaks at about 3.9, 4.8 and 25.90 2θ±0.20 20.

21. The crystalline form of valrubicin of claim 19, characterized by a Fourier-transform infrared spectrum having peaks at about 1724, 1415, and 1019 cm⁻¹.

22. The crystalline form of valrubicin of claim 19, characterized by a powder X-ray diffraction pattern as depicted in FIG. 1.

23. The crystalline form of valrubicin of claim 22, characterized by a Fourier-transform infrared spectrum as depicted in FIG. 2.

24. A process for preparing a crystalline form of valrubicin characterized by data selected from the group consisting of at least one of: a PXRD pattern having peaks at 3.9, 4.8 and 25.9° 2θ±0.2° 2θ; a PXRD pattern as depicted in FIG. 1; a FT-IR spectrum having peaks at about 1724, 1415, and 1019 cm⁻¹, and an FT-IR spectrum as depicted in FIG. 2 by providing a suspension of valrubicin in a mixture of a solvent selected from the group consisting of: dichloromethane, acetone, acetonitrile, methyl ethyl ketone, methylisobutyl ketone and an anti-solvent selected from the group consisting of: diisopropylether, and methyl-tert-butyl ether, and maintaining the suspension at a temperature of about 0° C. to 40° C. to obtain the crystalline form of valrubicin.

25. A pharmaceutical composition comprising a crystalline form of valrubicin characterized by data selected from the group consisting of at least one of: a powder X-ray diffraction pattern having peaks at 3.9, 4.8 and 25.9° 2θ±0.2° 2θ; a powder X-ray diffraction pattern as depicted in FIG. 1; a Fourier-transform infrared spectrum having peaks at about 1724, 1415, and 1019 cm⁻¹; and a Fourier-transform infrared spectrum as depicted in FIG. 2; and at least one pharmaceutically acceptable excipient.

26. A method of treating bladder cancer comprising administering a therapeutically effective amount of a pharmaceutical composition comprising a crystalline form of valrubicin characterized by data selected from the group consisting of at least one of: a powder X-ray diffraction pattern having peaks at 3.9, 4.8 and 25.9° 2θ±0.2° 2θ; a powder X-ray diffraction pattern as depicted in FIG. 1; a Fourier-transform infrared spectrum having peaks at about 1724, 1415, and 1019 cm⁻¹; and a Fourier-transform infrared spectrum as depicted in FIG. 2; and at least one pharmaceutically acceptable excipient to a patient in need thereof.
